(19) United States
(12) Patent Application Publication
(10) Pub. No.: US 2010/0047852 A1
(43) Pub. Date: Feb. 25, 2010

(54) SELECTIVE CULTURE MEDIUM

(75) Inventor: Michael Ford, Newcastle Upon Tyne (GB)

Correspondence Address: KNOBBE MARTENS OLSON & BEAR LLP, 2040 MAIN STREET, FOURTEENTH FLOOR, IRVINE, CA, 92614, US

(73) Assignee: NEWCASTLE UPON TYNE HOSPITALS NHS FOUNDATION TRUST, Newcastle Upon Tyne (GB)

(21) Appl. No.: 12/280,278

(22) PCT Filed: Feb. 23, 2007
(86) PCT No.: PCT/GB2007/000640

§ 371 Date: Nov. 10, 2009

(30) Foreign Application Priority Data
Feb. 24, 2006 (GB) 0603766.7

(51) Int. Cl. C12Q 1/04

(52) U.S. Cl. 435/34

(57) ABSTRACT

The present invention discloses a selective culture medium for detecting MRSA and selective drug or multidrug resistant microorganisms where the expression of said (multi)drug resistance is by virtue of the production by said microorganisms of β-lactamase enzymes. The medium comprises: (i) a broad spectrum antibiotic capable of selectively inhibiting growth/replication of (a) organisms that do not produce β-lactamase enzymes, (b) organisms that while producing β-lactamase do not exhibit an ESBL or AmpC phenotype and (c) Methicillin-susceptible S. aureus, and (ii) a compound capable of inducing production of β-lactamases in an organism, and which compound is included at a concentration that is subinhibitory for growth of said microorganism. Also provided are a method and kit for selectively isolating any MRSA and/or ESBL, or AmpC β-lactamase producing microorganisms present in a sample using the selective culture medium.

The present application is concerned with a selective culture medium and in particular a selective culture medium capable of detecting the presence of methicillin resistant Staphylococcus aureus (MRSA) and microorganisms capable of producing β-lactamases.

The Centre for Disease Control estimates that in the United States up to 2 million people suffer from nosocomial infections each year, and as a result, 90,000 patients die (1). These infections are becoming more difficult to treat due to the increased antimicrobial resistance associated with nosocomial bacterial pathogens. Methicillin resistant Staphylococcus aureus (MRSA) strains are now responsible for over 50% of all hospital acquired S. aureus infections in the US (1). Not only are MRSA strains causing high levels of nosocomial infection but other multi-drug resistant isolates are established causes of nosocomial infection. Of major concern are Gram-negative bacilli; particularly Enterobacteriaceae strains which produce extended spectrum β-lactamases (ESBLs) or hyper produced chromosomal or plasmid-mediated AmpC β-lactamases.

ESBLs destroy cephalosporins and other β-lactam agents that are often given as first line therapeutic agents to severely ill patients. Inappropriate treatment of severe infections caused by these strains is associated with an increased morbidity and mortality. A second major concern is that detection of antimicrobial resistance can be problematic with these strains as resistance to cephalosporins is not always obvious in the laboratory. As such, inappropriate therapy is delayed with often serious sequelae.

Enterobacteriaceae that produce AmpC β-lactamases are a second group of multi drug resistant Gram-negative bacilli, which are a major cause of nosocomial infection. AmpC production is associated with a wide range of bacterial genera including Enterobacter spp., Citrobacter spp., Serratia spp. and Morganella spp (6). In one study sepsis caused by Serratia sp had a 30-day mortality rate of 50%. As with ESBL-producing Enterobacteriaceae, detection of resistance mediated by AmpC β-lactamase can be problematic in vitro, which can result in a delay before optimal therapy is administered. This delay can impact on patient morbidity and mortality.

There are numerous culture media which are commonly used for the selective isolation of MRSA strains, examples of which include Baird Parker medium plus ciprofloxacin (2), Mannitol Salt agar plus Oxacillin (3) and more recently various chromogenic agars. These are based on the use of a chromogenic compound that when hydrolysed by S. aureus strains releases a coloured product. By incorporating cefoxitin as the selective agent only S. aureus strains resistant to cefoxitin (a surrogate for methicillin) are capable of growth and producing a coloured product (4). The principle of using cefoxitin in laboratory media for the selective isolation of MRSA is now well established (4, 5). These media typically contain other selective agents to inhibit the growth of non-pathogenic staphylococci, enterococci and Gram-negative organisms. The presence of a high concentration of cefoxitin in these media would preclude their use for detection of ESBL producing strains because such organisms are, typically, susceptible to cefoxitin. These media are, therefore, only targeted for the selective isolation of MRSA strains.

Selective culture media for the isolation of ESBL producing strains are very limited. In most situations the organisms are isolated from clinical samples on routine non-selective media, and the ESBL phenotype determined by use of specific antibiotic discs. These discs contain a third generation cephalosporin e.g. Cefpodoxime and a second disc contains the same cefpodoxime plus clavulanic acid. The detection of enhanced susceptibility to Cefpodoxime/Clavulanic Acid when directly compared with cefpodoxime alone, indicates the likely presence of an ESBL.

To date the only reported culture medium designed specifically for the detection of ESBL-producing Enterobacteriaceae is produced by AES and distributed in the UK by Microgen Bioproducts. The medium contains two distinct media in the same Petri dish; Drigalski agar plus Cefotaxime on one side and MacConkey plus Ceftazidime on the other. Growth of Enterobacteriaceae on either medium is suggestive of the strain being a producer of ESBL. Currently there are no selective isolation media for the specific isolation of AmpC β-lactamase producing strains from clinical samples.

Whilst the AES ESBL medium contains two third generation cephalosporins, no medium exists for the selective isolation of both ESBL and AmpC phenotypes on the same plate.

The present inventors have now developed a selective culture medium specifically designed for the simultaneous detection of MRSA and particular bacterial strains, that exhibit multidrug resistance by virtue of their production of β-lactamases, which may be either ESBL or AmpC β-lactamases.

Therefore, according to a first aspect of the present invention, there is provided a selective culture medium for detecting MRSA and selective drug or multidrug resistant microorganisms where the expression of said (multi)drug resistance is by virtue of the production by said microorganisms of β-lactamase enzymes, said medium comprising

(i) a broad spectrum antibiotic capable of selectively inhibiting growth/replication of (a) organisms that do not produce β-lactamase enzymes, (b) organisms that while producing β-lactamase do not exhibit an ESBL or AmpC phenotype and (c) Methicillin-susceptible S. aureus, and

(ii) a compound capable of inducing production of β-lactamases in an organism, and which compound is included at a concentration that is subinhibitory for growth of said microorganism.

The culture medium according to the invention, therefore, includes a broad spectrum antibiotic as a sole selective agent. This agent, advantageously, results in the selective inhibition of the vast majority of microorganisms, including the Enterobacteriaceae. The broad spectrum antibiotic advantageously inhibits growth/replication of organisms that do not produce either an ESBL or AmpC β-lactamase, and these enzymes are responsible for drug resistance in a number of micro organisms. Many organisms, however, also produce β-lactamase enzymes but not of the ESBL or AmpC type and these confer some degree of antimicrobial resistance. These micro organisms are also therefore inhibited by virtue of the broad spectrum antibiotic. Therefore, colonies resembling micro organisms, such as the Enterobacteriaceae, that grow on this medium, are considered to be resistant to β-lactam antibiotics by production of an ESBL or AmpC phenotype. If antibiotic treatment is indicated to eradicate such strains, β-lactam agents can therefore be avoided. The culture medium may, therefore, be utilised alongside conventional media to confirm to clinicians that an individual from whom the sample is taken is infected with a multidrug resistant strain. Knowing that an individual has a multidrug resistant strain assists in the management of the patient. Knowing that the patient has such a multidrug resistant strain will alter the antibiotic management of the patient and initiate control of infection investigations twenty four hours sooner than at present and which will have a significant impact in limiting the spread of the multidrug resistant organisms to other individuals. As of yet, no single medium exists that permits such information to be obtained at such an early stage.

The culture medium, according to this aspect of the invention permits the detection of those organisms that can demonstrate an ESBL or AmpC phenotype under the appropriate conditions. Strains that are capable of producing this enzyme do not always do so and enzyme production is frequently ‘repressed’ thus giving an erroneous indication of the organism's ability to demonstrate antibiotic resistance. The presence of the broad spectrum antibiotic inhibits a significant proportion of Enterobacteriaceae and other organisms, many of which are capable of producing β-lactamases.

Suitable inducing compounds include any β-lactam antibiotic in an appropriate concentration, including for example Clavulanic acid, Imipenem, Cefoxitin, Meropenem, Ertapenem, Amoxicillin, Penicillin, co-Amoxyclav, Aztreonam, Cefotaxime, Ceftazidime, Ceftriaxone, Cefepime, Cefixime, Cefpirome, Cefotetan, Ceftibuten, Cefprozil, Sulbactam, Methicillin, Oxacillin, Meropenem, Cephlexin, Cefalothin or Cefuroxime, all of which compounds can be obtained commercially

The concentration of the inducer is kept low as this agent may inhibit the growth of ESBL-producing Enterobacteriaceae at a concentration regularly used in other selective media. For example, Cefoxitin is a well recognized agent that is able to induce production of AmpC β-lactamase in Enterobacteriaceae. However, as aforementioned, many ESBL strains are susceptible to Cefoxitin, which could initially preclude its use a selective agent for ESBL producing strains.

In a preferred embodiment, the compound capable of inducing β-lactamase production, which may preferably be Cefoxitin, is included at a concentration of up to 1 mg/L and preferably at a concentration of up to 0.5 mg/L and more preferably up to 0.1 mg/L. Preferably, the broad spectrum antibiotic is included at a concentration of from 1 to 16 mg/L, and even more preferably at a concentration of 4 mg/L.

The nutrients of the basal medium are not critical to its performance and any peptone-based medium that readily supports the growth of S. aureus and Enterobacteriaceae for example, tryptone soy agar, Isosensitest agar or Nutrient agar amongst others, may be used. In one embodiment, Columbia agar may be used but any nutrient source may be utilised with equal effectiveness. The medium may also be supplemented with blood, which advantageously enhances growth and differentiation of colony types. In a preferred embodiment, any animal blood, such as sheep, goat or horse blood may be used at a concentration of, for example, 5% v/v.

In a preferred embodiment of the invention, the broad spectrum antibiotic comprises any β-lactam agent capable of being hydrolysed by both ESBL and AmpC β-lactamases. Preferably, said antibiotic is selected from any suitable β-lactam such as Aztreonam, Cefotaxime, Ceftazidime, Ceftriaxone, Cefepime, Cefixime, Cefpirome, Cefotetan, Ceftibuten, Cefprozil, Meropenem or Ertapenem. However, in a preferred embodiment Cefpodoxime is used as the selective agent. In one embodiment, a different inducing β-lactamase is utilised than the broad spectrum antibiotic of the invention. This, advantageously, inhibits the growth of Methicillin-susceptible S. aureus, most Enterobacteriaceae that do not produce ESBLs or AmpC β-lactamases and a variety of other bacteria, including most strains of Streptococci, Haemophillus spp., coagulase negative staphylococci and Neisseria spp. Other than the target organisms, some bacteria may not be inhibited including strains of Acinetobacter spp., Pseudomonas spp., Stenotrophomonas maltophillia and strains of Klebsiella oxytoca that show hyper production of K1 chromosomal β-lactamase. In addition, to the target strains these organisms have been responsible for various nosocomial outbreaks of infection and frequently demonstrate antibiotic resistance. Therefore, the medium can, advantageously, be used for general screening for bacteria with antimicrobial resistance.

Methicillin-susceptible strains of S. aureus (i.e, non-MRSA) will not grow on the medium of the invention. Thus, any colonies confirmed as S. aureus may be regarded as MRSA unless proven otherwise by subsequent tests. This has beneficial implications for antimicrobial chemotherapy, such as the avoidance of inappropriate treatment with β-lactam agents, quinolones and macrolides against all of which MRSA commonly expresses resistance. The use of antibiotic resistance such as resistance to Cefpodoxime, for example, as a diagnostic marker for ESBL-producing Enterobacteriaceae, is well recognised in the art (6). However, there has not yet been any description of the use of such antibiotics in a selective medium for isolation of MRSA together with a ESBL or AmpC β-lactamase inducing compound.

In one embodiment, selectivity may be increased further by the addition of, for example, an antifungal agent, to inhibit yeasts and other fungi. In one embodiment said anti-fungal agent comprises Amphotericin.

The selective medium according to the invention is particularly advantageous, because it permits direct application of a clinical sample taken from a patient onto the medium and after an overnight incubation visualisation can determine if an organism, such as MRSA or β-lactamase producing organisms exhibiting ESBL or AmpC phenotype are present. Once present on the medium it is a routine matter to then ascertain the type of organisms present.

Visualisations may therefore be carried out merely by inspection. Alternatively, the medium may also include chromogenic substrates. Chromogenic substrates specific for MRSA and Enterobacteriaceae can be used. Suitable substrates include chromogenic substrates for sand β-galactosidase, glucuronidase, glucosidase and phosphatase activity.

According to a further aspect of the invention there is also provided, a method of selectively isolating any MRSA and/or ESBL or AmpC β-lactamase producing microorganisms present in a sample, said method comprising applying said sample to a culture medium according to the invention wherein any growth of bacteria on said medium is indicative of the presence of either MRSA and/or ESBL or AmpC β-lactamase producing microorganisms in said sample.

In an even further aspect of the invention, there is also provided a kit for selectively isolating any MRSA and/or ESBL or AmpC β-lactamase producing microorganisms present in a sample, said kit comprising a culture medium according to the invention and means to contact said culture medium with a sample to be tested.

The invention will now be further described with reference to the following exemplary embodiments.

### EXAMPLES

Medium Preparation.

The basal medium was prepared by adding 40 g of Columbia agar base to 950 ml of sterile de-ionised water and mixed to dissolve. After dissolving the medium was autoclaved at 121° C. for 15 minutes and cooled to 45° C. Stock solutions (prepared in sterile distilled water) of both cefpodoxime and cefoxitin were added to give various concentrations of both agents and the medium was mixed thoroughly. Finally 50 ml of sterile horse blood was added and the medium again mixed thoroughly before pouring into sterile Petri dishes and allowed to solidify.

Various batches of agar were prepared containing the following concentrations of antibiotics:

Batch 1: Cefpodoxime 4 mg/L plus Cefoxitin 0.1 mg/L

Batch 2: Cefpodoxime 4 mg/L plus Cefoxitin 0.2 mg/L

Batch 3: Cefpodoxime 4 mg/L plus Cefoxitin 0 mg/L

Batch 4: No antimicrobials (Growth control).

Inoculation of the Medium with Test Strains.

A range of both clinical isolates and strains obtained from Type-culture collections were prepared at a concentration equivalent to McFarland standard 0.5. Using a multi-point inoculator 1 ml of each suspension was inoculated onto the test media containing the various concentrations of antibiotics. Plates were incubated aerobically at 30 and 37° C. and examined for growth after overnight incubation.

The results are shown in table 1.

TABLE 1
Growth of 117 bacterial strains
inoculated onto the test media. Figures are expressed as a percentage of strains
growing on the media

| CPD only | CPD/Fox 0.1 | CPD/Fox 0.2 | CPD only | CPD/Fox | CPD/Fox |
|---|---|---|---|---|---|
| (37° C.) | (37° C.) | (37° C.) | (30°) | 0.1 (30° C.) | 0.2 (30° C.) |

S. Aureus

| | | | | | | |
|---|---|---|---|---|---|---|
| MRSA | 93 | 81 | 79 | 100 | 100 | 100 |
| (42) | | | | | | |
| MSSA | 0 | 0 | 0 | 0 | 0 | 0 |
| (18) | | | | | | |

Enterobacteriaceae

| | | | | | | |
|---|---|---|---|---|---|---|
| ESBL | 100 | 100 | 100 | 100 | 100 | 93 |
| (15) | | | | | | |
| AmpC | 50 | 79 | 86 | 43 | 86 | 86 |
| (14) | | | | | | |
| Non AmpC | 0 | 0 | 0 | 0 | 0 | 0 |
| (25) | | | | | | |
| K. oxytoca KI | 100 | 100 | 100 | 100 | 1 (weak) | 0 |
| (1) | | | | | | |

Other Gram negatives

| | | | | | | |
|---|---|---|---|---|---|---|
| Acinetobacter | 100 | 100 | 100 | 100 | 100 | 100 |
| sp | | | | | | |
| (1) | | | | | | |
| P. aeruginosa | 100 | 100 | 100 | 100 | 100 | 100 |
| (1) | | | | | | |

All strains grew well on media without antimicrobials at both temperatures.

Further Evaluation with a Range of ESBL Producing Strains.

A collection of 41 ESBL producing isolates was prepared in sterile de-ionised water to an equivalent of McFarland 0.5. Using a multi-point inoculator 1 μl of each suspension was inoculated onto a test medium containing Cefpodoxime and Cefoxitin at final concentrations of 4 and 0.1 mg/l respectively. Plates were incubated aerobically at 30° C. and examined for growth after overnight incubation.

Results.

Growth on the test medium occurred with 39 of the 41 strains (95%).

Use of Medium for the Detection of MRSA, ESBL and AmpC Strains Directly from Respiratory Samples.

Test medium containing Cefpodoxime and Cefoxitin at final concentrations of 4 and 0.1 mg/L respectively were inoculated with 10 μl of diluted homogenised sputum or neat sputum depending upon the sample type. In addition “routine” culture plates were also inoculated, these comprised blood, chocolate, and chocolate plus Bacitracin plates. Test media were incubated at 30° C. in air and routine culture plates in 5% CO₂ Growth on routine plates was compared with growth on the test medium and the following protocol applied.

A total of 433 sputum samples were cultured onto the test medium and routine culture media. Test media were incubated at 30° C. in air and examined after overnight incubation. Routine culture plates were examined after overnight incubation in 5% CO₂ and again after 48 h incubation.. The results using the test media were compared with routine sputum culture results. The routine culture plates were read independently of the test media by a different Biomedical Scientists.

Suspect colonies of S. aureus on the test media were tested with Slidex Staph Plus latex reagent to confirm their identity as S. aureus. A sensitivity test was then performed using standard methods to determine if the isolate was resistant to methicllin.

Colonies of Gram negative organisms on the test media were initially tested for the presence of cytochrome oxidase activity and a susceptibility test performed according to BSAC methodology. In addition the isolate was identified by the LOGIC identification system, and if required by API 20E to determine if the isolate was typically associated with AmpC production. The antimicrobial susceptibility test protocol contained both Cefpodoxime and Cefpodoxime plus clavulanic acid discs to determine if the isolate was a producer of ESBL. Results are shown below.

### Results—Routine Sputum Plates

251 showed no growth or contained regional flora on routine media.

46 contained a conventional respiratory pathogen (e.g. Haemophilus influenzae, Moraxella catarrhalis or Streptococcus pneumoniae).

60 grew a strain of Enterobacteriaceae.

31 grew Pseudomonas sp.

33 grew S. aureus

11 grew yeast

1 grew pure growth of a haemolytic streptococcus.

Results—Test Media.

325 plates showed no growth (or scanty normal flora e.g. alpha haemolytic streptococci).

44 plates grew presumptive Enterobacteriaceae

23 plates grew S. aureus

29 plates grew Pseudomonas sp

12 plates grew yeast

### CONCLUSION

All S. aureus strains (23) growing on the test medium were confirmed as MRSA strains using standard susceptibility and identification tests. Of the 44 Gram negative organisms growing on the test medium, 2 strains were K1 hyper-β-lactamase producing K. oxytoca, 20 strains were S. marcescens, 11 E. cloacae, 3 Acinetobacter sp, and 6 S. maltophillia strains. Two strains were found to be ESBL producing.

Of the 16 Enterobacteriaceae strains that failed to grow on the test medium all were susceptible to cefuroxime and none showed an AmpC or ESBL phenotype. The strains were E. coli (n=4), K. pneumoniae (n=7), K. oxytoca (n=3), and Proteus sp (n=2).

Except for two strains of K1 hyper-β-lactamase producing K. oxytoca, all Enterobacteriaceae growing on the test media were identified as strains which showed an ESBL or AmpC phenotype.

### REFERENCES

1) Diekema D J, Dodgson K J, Sigurdardottir B, Pfaller M A. Rapid detection of antimicrobial-resistant organism carriage: an unmet clinical need. J Clin Microbiol. 2004 July; 42(7):2879-83.

2) Bowers K M, Wren M W, Shetty N P. Screening for methicillin resistance in Staphylococcus aureus and coagulase-negative staphylococci: an evaluation of three selective media and Mastalex-MRSA latex agglutination. Br J Biomed Sci. 2003;60(2):71 -4.

3) Dias C G, Ropke M V, Superti S, Berquo L, d'Azevedo P. Use of a novel selective medium to detect methicillin-resistant Staphylococcus aureus in colonized patients of an intensive care unit. Infect Control Hosp Epidemiol. 2004 February;25(2): 130-2.

4) Perry J D, Davies A, Butterworth L A, Hopley A L, Nicholson A, Gould F K. Development and evaluation of a chromogenic agar medium for methicillin-resistant Staphylococcus aureus. J Clin Microbiol. 2004 October;42(10):4519-23.

5) Smyth R W, Kahlmeter G. Mannitol salt agar-cefoxitin combination as a screening medium for methicillin-resistant Staphylococcus aureus. J Clin Microbiol. 2005 August; 43(8):3797-9.

6) Livermore D M, Brown D F. Detection of beta-lactamase-mediated resistance. J Antimicrob Chemother. 2001 July; 48 Suppl 1:59-64.

7) Huber T W, Thomas J S. Detection of resistance due to inducible beta-lactamase in Enterobacter aerogenes and Enterobacter cloacae. J Clin Microbiol. 1994 October; 32(1 0):248 1-6.

1. A selective culture medium for detecting MRSA and drug or multidrug resistant microorganisms where the expression of said (multi)drug resistance is by virtue of the production by said microorganisms of ESBL and/or AmpC enzymes, said medium comprising;
(i) a broad spectrum antibiotic capable of selectively inhibiting growth/replication of (a) organisms that do not produce β-lactamase enzymes, (b) organisms that while producing β-lactamase do not exhibit an ESBL or AmpC phenotype and (c) methicillin-susceptible S. aureus, and
(ii) a compound capable of inducing production of β-lactamases in an organism, the compound being selected from the group consisting of Imipenem, Cefoxitin, Meropenem, Ertapenem, Amoxicillin, Penicillin, Co-Amoxyclav, Aztreonam, Cefotaxime, Ceftazidime, Ceftriaxone, Cefepime, Cefixime, Cefpirome, Cefotetan, Ceftibuten, Cefprozil, Cephlexin, Cefalothin, Sulbactam, Methicillin, Oxacillin and Cefuroxime, and which compound is included at a concentration of up to about 0.5mg/L and which is subinhibitory for growth of said microorganism, and
wherein the compound capable of inducing production of β-lactamase is different from the broad spectrum antibiotic.

2. The culture medium of claim 1, wherein said broad spectrum antibiotic is provided at a concentration of from 1 to 16 mg/L.

3. The culture medium of claim 1, wherein said broad spectrum antibiotic is provided at a concentration of about 4 mg/L.

4. (canceled)

5. The culture medium of claim 1 further comprising an antifungal agent.

6. The culture medium of claim 1, wherein said broad spectrum antibiotic comprises any β-lactam agent capable of being hydrolysed by ESBLs and AmpC β-lactamases.

7. The culture medium of claim 1, wherein said broad spectrum antibiotic is selected from the group consisting of Aztreonam, Cefotaxime, Ceftazidime, Ceftriaxone, Cefepime, Cefixime, Cefpirome, Cefotetan, Ceftibuten, Cefprozil, Meropenem, Ertapenem and Cefpodoxime.

8. The culture medium of claim 7, wherein said broad spectrum antibiotic is Cefpodoxime.

9. The culture medium of claim 1, wherein said compound capable of inducing β-lactamase is any β-lactam antibiotic.

10. (canceled)

11. The culture medium of claim 1, wherein said medium comprises agar.

12. The culture medium according to claim 11, wherein said agar is a nutrient based medium.

13. The culture medium of claim 1, wherein said medium is further enhanced with blood or other nutrients.

14. The culture medium according to claim 13, wherein said blood is animal blood.

15. The culture medium of claim 13, wherein said blood is provided at a concentration of 5% v/v.

16. (canceled)

17. A method of selectively isolating any MRSA and/or ESBL or AmpC β-lactamase producing microorganisms present in a sample, said method comprising applying said sample to the culture medium of claims of claim 1 wherein any growth of bacteria on said medium is indicative of the presence of either MRSA and/or ESBL or AmpC β-lactamase producing microorganisms in said sample.

18. A kit for selectively isolating any MRSA and/or AmpC or ESBL β-lactamase producing microorganisms present in a sample, said kit comprising the culture medium of claim 1 and means to contact said culture medium with a sample to be tested.

19. The culture medium of claim 12, wherein said nutrient based medium is selected from the group consisting of tryptone soy agar, Isosensitest agar, nutrient agar and Columbia agar.

20. The culture medium of claim 14, wherein said animal blood is selected from the group consisting of horse blood, sheep blood and goat blood.

21. A method of selectively isolating any MRSA and/or ESBL or AmpC β-lactamase producing microorganisms present in a sample, said method comprising applying said sample to a culture medium according to claim 1, wherein any growth of bacteria on said medium is indicative of the presence of either MRSA and/or ESBL or AmpC β-lactamase producing microorganisms in said sample.

22. A kit for selectively isolating any MRSA and/or AmpC or ESBL β-lactamase producing microorganisms present in a sample, said kit comprising a culture medium according to claim 1 and means to contact said culture medium with a sample to be tested.
